(19) United States
(12) Patent Application Publication
(10) Pub. No.: US 2010/0260842 A1
(43) Pub. Date: Oct. 14, 2010

(54) PSEUDOEPHEDRINE PHARMACEUTICAL FORMULATIONS

(76) Inventors: Rashmi Nair, Indore (IN); Praveen Raheja, Janakpuri (IN); Sanjay Chhagan Wagh, Nashik (IN); Raviraj Sukumar Pillai, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 12/755,374

(22) Filed: Apr. 6, 2010

Related U.S. Application Data
(60) Provisional application No. 61/220,260, filed on Jun. 25, 2009.

(30) Foreign Application Priority Data
Apr. 6, 2009 (IN) 805/CHE/2009

(51) Int. Cl. A61K 9/28; A61K 9/14; A61K 31/137; A61P 11/02; A61P 11/14; A61P 11/00

(52) U.S. Cl. 424/472; 424/490; 424/492; 424/494; 424/495; 514/653

(57) ABSTRACT

Controlled-release pharmaceutical formulations comprising pseudoephedrine or any of its pharmaceutically acceptable salts, processes for preparing the pharmaceutical formulations, and methods of using the formulations.

### INTRODUCTION

Aspects of the present invention relate to controlled-release pharmaceutical formulations comprising pseudoephedrine or any of its pharmaceutically acceptable salts. Aspects also relate to processes for preparing the pharmaceutical formulations and methods of using such formulations.

The drug compound having the adopted name “pseudoephedrine” has a chemical name (1S,2S)-2-methylamino-1-phenylpropan-1-ol, and can be represented by structural Formula I.

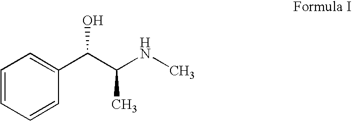

Pharmacologically, pseudoephedrine is a sympathomimetic amine. Pseudoephedrine is used as a bronchodilator and as a peripheral vasoconstrictor. Pseudoephedrine is indicated for temporary relief of nasal congestion due to the common cold, for temporary relief of nasal congestion associated with sinusitis, for relief of cough due to minor throat irritation as may occur with the common cold or inhaled irritants, for promoting nasal drainage, for promoting sinus drainage, for its help in loosening phlegm and bronchial secretion, for helping rid the bronchial passage of mucus, for relief of hay fever, and for the relief of upper respiratory allergies.

Pseudoephedrine is an active ingredient in numerous products, including various tablets for immediate release and controlled release of the drug. The drug is typically present in the form of a salt, such as the hydrochloride or sulfate. Among the commercially available products is SUDAFED™ 12 Hour and 24 Hour tablets, respectively containing 120 mg and 240 mg of pseudoephedrine hydrochloride and sold by McNeil-PPC, Inc. in the USA.

Pseudoephedrine hydrochloride occurs as fine, white to off-white crystals or powder, having a faint characteristic odor. It is very soluble in water, freely soluble in alcohol, and sparingly soluble in chloroform. The molecular weight is 201.70. The molecular formula is C₁₀H₁₅NO.HCl.

U.S. Pat. No. 5,895,663 discloses pseudoephedrine extended release tablets containing a sustained release hydrophilic matrix and microcrystalline cellulose as a disintegrant, formed by a direct compression method.

U.S. Pat. No. 5,451,409 discloses a dry mixed pseudoephedrine tablet in which a mixture of hydroxypropyl cellulose and hydroxyethyl cellulose forms the sustained release matrix, and 0.5-10% HPMC is present as a binder.

U.S. Pat. Nos. 5,558,879, 5,612,059, 6,500,459, 5,292,534, 5,451,409, 5,458,887, 5,885,615, 6,033,685, 6,245,351, 6,592,901, and 6,946,146, and U.S. Patent Application Publication Nos. 2004/0142035, 2006/0008527, 2007/0020335, and 2008/0095843 disclose compositions of pseudoephedrine or its salts. M. Ishida et al., “A Novel Approach to Sustained Pseudoephedrine Release: Diferentially Coated Mini-tablets in HPMC Capsules,” International Journal of Pharmaceutics, Vol. 359 (1-2), pp. 46-52, 2008, discloses capsules containing a mixture of separately formulated immediate release and sustained release mini-tablets, containing pseudoephedrine hydrochloride.

SUDAFED 24 Hour tablets are osmotic tablets, wherein a drug-containing tablet is surrounded by a semipermeable membrane and one or more lamina with or without drug. A laser drilled opening in the tablet acts as the vent for drug delivery. In the gastrointestinal tract, water enters the tablet and a contained osmotic agent swells to exert the pressure required for the controlled release of the drug through the opening. There are various modifications that are available for this technology. However, formulating an osmotic drug delivery system presents challenges, such as the specialized equipment required for laser drilled holes and multi-step processing for making tablet layers and coatings.

There is a need for simple and cost-effective drug delivery technology for pseudoephedrine that provides the therapeutic advantages of an osmotic delivery system.

### SUMMARY

The present invention relates to controlled release pharmaceutical formulations comprising pseudoephedrine or its pharmaceutically salts, having a controlled drug release profile with an immediate release phase, followed by drug delivery at a controlled rate over a period of time.

In embodiments, the invention includes controlled release pharmaceutical formulations having:

1) a core comprising:
a) 20 to 50% of pseudoephedrine or its salt;
b) 20 to 50% of a rate controlling polymer;
c) 1 to 10% of binder; and
d) optionally, other pharmaceutical excipients; and

2) a coating comprising:
a) 1 to 15% of pseudoephedrine or its salt;
b) 1 to 25% of a rate controlling polymer;
c) 1 to 15% of a pore forming agent; and
d) 1 to 10% of a plasticizer.

In embodiments, the invention includes controlled-release pharmaceutical formulations having:

1) a core comprising:
a) 20 to 50% of pseudoephedrine or its salt;
b) 20 to 50% of a hydrophilic polymer;
c) 1 to 10% of a water soluble binder; and
d) optionally, other pharmaceutical excipients; and

2) a coating comprising:
a) 1 to 15% of pseudoephedrine or its salt;
b) 1 to 25% of a hydrophobic polymer;
c) 1 to 15% of a water soluble polymer; and
d) 1 to 10% of a water insoluble plasticizer.

In embodiments, the invention includes controlled-release pharmaceutical formulations having:

1) a core comprising:
a) 20 to 50% of pseudoephedrine hydrochloride;
b) 20 to 50% of a hydrophilic polymer;
c) 1 to 10% of a water-soluble binder; and
d) optionally, other pharmaceutical excipients; and

2) a coating comprising:
a) 1 to 15% of pseudoephedrine hydrochloride;
b) 1 to 25% of a hydrophobic polymer;
c) 1 to 15% of a water soluble polymer; and
d) 1 to 10% of a plasticizer.

In embodiments, the invention includes controlled-release pharmaceutical formulations having:

1. a core comprising:
a) 10 to 40 percent by weight of pseudoephedrine or a salt thereof;
b) 5 to 30 percent by weight of a water soluble polymer;
c) 1 to 10 percent by weight of a binder;
d) 15 to 40 percent by weight of a water insoluble filler; and
e) optionally, one or more other pharmaceutical excipients; and

2. a coating comprising:
a) 1 to 15 percent by weight of pseudoephedrine or a salt thereof;
b) 1 to 25 percent by weight of a combination of a water soluble polymer and a water insoluble polymer; and
c) 0.1 to 15 percent by weight of a water insoluble plasticizer.

In embodiments, the invention includes controlled-release pharmaceutical formulations having:

I. a core containing about 50-90 percent of a total amount of pseudoephedrine in the formulation, comprising:
a) 10 to 40 percent by weight of pseudoephedrine or a salt thereof;
b) 5 to 30 percent by weight of a water soluble polymer;
c) 1 to 10 percent by weight of a binder;
d) 15 to 40 percent by weight of a water insoluble filler; and
e) optionally, one or more other pharmaceutical excipients; and

II. a coating containing about 10-50 percent of a total amount of pseudoephedrine in the formulation, comprising:
a) 1 to 15 percent by weight of pseudoephedrine or a salt thereof;
b) 1 to 25 percent by weight of a combination of a water soluble polymer and a water insoluble polymer; and

0.1 to 15 percent by weight of a water insoluble plasticizer.

Further embodiments of the invention include methods of preparing pharmaceutical formulations containing pseudoephedrine or its salts.

In embodiments, the invention includes methods of using pharmaceutical formulations containing pseudoephedrine or its salts.

### DETAILED DESCRIPTION

Percentages are expressed herein on a weight basis, unless the context indicates otherwise.

Aspects of the present invention relate to pharmaceutical formulations comprising pseudoephedrine or pharmaceutically acceptable salts thereof. Further aspects relate to controlled-release pharmaceutical formulations comprising pseudoephedrine or pharmaceutically acceptable salts thereof.

Pseudoephedrine is a sympathomimetic amine, and its principal mechanism of action relies on indirect action on the adrenergic receptor system. While it may have weak agonist activity at α- and β-adrenergic receptors, the principal mechanism is to cause the release of endogenous norepinephrine (noradrenaline) from storage vesicles in presynaptic neurons. The displaced noradrenaline is released into the neuronal synapse where it is free to activate the postsynaptic adrenergic receptors.

As used herein, the term “pseudoephedrine” includes the compound pseudoephedrine and any of its pharmaceutically acceptable salts, polymorphs, solvates, esters, hydrates, enantiomers, racemates, and mixtures thereof.

As used herein, the term “controlled release” indicates a formulation that releases drug in a manner different from that of an immediate release dosage form (in which drug release commences promptly in the stomach after oral administration, and continues at a rapid rate as the dosage form disintegrates), such as providing a drug release that is delayed for a desired time period after administration, and/or a drug release that occurs during a desired time period after administration. The terms ‘sustained’, ‘extended’, ‘delayed’, ‘modified’, or ‘prolonged’ are included within the term “controlled,” and as applied include any drug delivery system which achieves the release of drug after and/or over a desired period of time, e.g., providing dosage forms that can be administered every 8 hours, 12 hours, 24 hours, etc. The drug delivery systems include those such as modified-release matrix cores, modified-release matrix cores coated with a release-modifying coating, enteric coated dosage forms, dosage forms surrounded by a release-modifying coating, gastro-retentive dosage forms, muco-adhesive dosage forms, osmotic release dosage forms, etc. Immediate release pseudoephedrine products typically require dosing at 4-6 hour intervals, to provide the desired therapeutic effect.

The terms “dosage form” or “pharmaceutical formulation” as used herein relate to solid orally administered units comprising a drug and one or more pharmacologically inert excipients, such as tablets, mini-tablets, caplets, capsules, granules, pills, powders, microparticles, nanoparticles, etc.

The term “core” as used herein is defined to mean a particle or a solid vehicle, into which, or onto which, pseudoephedrine is uniformly or non-uniformly dispersed. A drug-containing core will desirably be formed by methods and materials well known in the art, such as for example by compressing, fusing, or extruding pseudoephedrine alone or together with at least one pharmaceutically acceptable excipient, or coating a pseudoephedrine composition onto a pharmacologically inert substrate.

“Rate-controlling” for purposes of the present invention refers to any polymer, or any generally water-insoluble substance, that acts to modulate the release of pseudoephedrine or its salts from a formulation, following immersion of a dosage form into an aqueous medium.

Rate-controlling polymers may be hydrophilic, hydrophobic, or lipophillic. Various rate controlling substances that are useful in the present invention include, but are not limited to, cellulose derivatives such as cellulose acetates, hydroxypropyl celluloses, carboxymethyl celluloses, methylcelluloses, ethylcelluloses, propylcelluloses, hydroxyethyl celluloses, carboxyethyl celluloses, carboxymethyl hydroxyethyl celluloses, hydroxymethyl celluloses, cellulose acetate pthalates, hydroxypropyl methylcellulose acetate succinates (HPMCAS), cellulose acetate trimellitates, carboxymethylethylcelluloses, and hydroxypropyl methylcelluloses (hypromelloses, or HPMC, including different grades such as Methocel K 4M, Type 2208, Methocel E 4M Type 2910, supplied by Colorcon Asia Private Limited), hydroxypropyl methyl phthalates, methacrylic acid-based polymers (including methacrylic acid-acrylic acid copolymers), such as those sold by Evonik Industries, Germany as Eudragit™ RL, Eudragit RS, Eudragit NE, Eudragit S, and Eudragit L, polyvinylpyrrolidones, polyalkylene glycols such as polyethylene glycol, vinyl acetate copolymers, polysaccharides (such as alginic acid, sodium alginate, xanthan gum and the like), polyethylene oxides, methacrylic acid copolymers, maleic anhydride-methylvinyl ether copolymers and derivatives and mixtures thereof, high molecular weight polyvinylalcohols, carbomers (e.g., Carbopol™ products), waxes such as fatty acids and glycerides, and any mixtures thereof.

In embodiments, the invention includes controlled-release compositions comprising pseudoephedrine or its salts, wherein the compositions are in monolithic or multi-particulate form.

Multi-particulate systems may be formed either by layering the drug onto pharmacologically inert cores, or by layering the drug together with rate-controlling polymers.

Non-limiting examples of various substances that can be used as the inert cores include insoluble inert materials such as glass particles or beads or silicon dioxide, non-pareil sugar seeds, calcium phosphate dihydrate, dicalcium phosphate, calcium sulfate dihydrate, microcrystalline celluloses (e.g., spherical Celphere™ products sold by Asahi Kasei Corporation, Tokyo, Japan in various particle sizes), cellulose derivatives, calcium carbonate, dibasic calcium phosphate anhydrous, dibasic calcium phosphate monohydrate, tribasic calcium phosphate, magnesium carbonate, magnesium oxide, soluble materials such as sugar spheres having sugars like dextrose, lactose, anhydrous lactose, spray-dried lactose, lactose monohydrate, mannitol, starches, sorbitol, and sucrose, insoluble inert polymeric materials such as spherical or nearly spherical core beads of polyvinylchloride or polystyrene, any other pharmaceutically acceptable insoluble synthetic materials, and mixtures thereof.

In embodiments, the invention includes controlled-release compositions comprising pseudoephedrine or its salts, having an immediate release fraction and a extended release fraction of pseudoephedrine or its salts. It is not necessary to have the same pseudoephedrine compound in both fractions.

In embodiments, about 50-90% of pseudoephedrine contained in a pharmaceutical formulation is provided in an extended release fraction, and the remaining pseudoephedrine is provided in an immediate release fraction. In particular embodiments, about 180 mg of contained pseudoephedrine is provided in an extended release fraction, and about 60 mg of the pseudoephedrine is provided in an immediate release fraction.

In embodiments, the invention includes controlled-release compositions comprising pseudoephedrine or its salts, wherein release of pseudoephedrine or its salts is by diffusion or by erosion.

In embodiments, the invention includes controlled-release pharmaceutical formulations having:

1) a core comprising:
a) 20 to 50% of pseudoephedrine or its salt;
b) 20 to 50% of a rate controlling polymer;
c) 1 to 10% of a binder; and
d) optionally, other pharmaceutical excipients; and

2) a coating comprising:
a) 1 to 15% of pseudoephedrine or its salt;
b) 1 to 25% of a rate controlling polymer;
c) 1 to 15% of a pore forming agent; and
d) 1 to 10% of a plasticizer.

In embodiments, the invention includes controlled-release pharmaceutical formulations having:

1) a core comprising:
a) 20 to 50% of pseudoephedrine or its salt;
b) 20 to 50% of a hydrophilic polymer;
c) 1 to 10% of a water soluble binder; and
d) optionally, other pharmaceutical excipients; and

2) a coating comprising:
a) 1 to 15% of pseudoephedrine or its salt;
b) 1 to 25% of a hydrophobic polymer;
c) 1 to 15% of a water soluble polymer; and
d) 1 to 10% of a water insoluble plasticizer.

In embodiments, the invention includes controlled-release pharmaceutical formulations having:

1) a core comprising:
a) 20 to 50% of pseudoephedrine hydrochloride;
b) 20 to 50% of a hydrophilic polymer;
c) 1 to 10% of a water soluble binder; and
d) optionally, other pharmaceutical excipients; and
2) a coating comprising:
a) 1 to 15% of pseudoephedrine hydrochloride;
b) 1 to 25% of a hydrophobic polymer;
c) 1 to 15% of a water soluble polymer; and
d) 1 to 10% of a plasticizer.

Pharmaceutical formulations comprising pseudoephedrine or its salts of the present invention can also comprise any number of inactive ingredients or excipients such as, but not limited to, any one or more of diluents, binders, disintegrants, glidants, lubricants, antioxidants, solvents, film-forming agents, and other adjuvants. Desirably, these ingredients are chemically and physically compatible with the pseudoephedrine or its salts.

Various useful fillers or diluents include, but are not limited to, starches available in different grades such as starch 1500, starch 1500 LM grade (low moisture content grade) from Colorcon, fully pregelatinized starches (e.g., National 78-1551 from Essex Grain Products), lactose in its different grades (e.g., Flowlac™ from Meggle Products and Pharmatose™ available from DMV), celluloses and derivatives thereof, such as Avicel™ PH101, PH102, PH301, PH302 and PHF20, and PH112 etc., confectioners sugar, carmellose, sugar alcohols such as mannitol (e.g., Pearlitol™ SD200), sorbitol, xylitol, calcium carbonate, magnesium carbonate, dibasic calcium phosphate dihydrate, and tribasic calcium phosphate.

Various useful binders include, but are not limited to, hydroxypropylcelluloses (Klucel™ LF and Klucel EXF) hydroxypropyl methylcelluloses (Methocel™), polyvinylpyrrolidones or povidones (PVP-K25, PVP-K29, PVP-K30, PVP-K90), Plasdone™ S630 (copovidone), powdered acacia, gelatin, guar gum, carbomers (e.g. Carbopol™), methylcelluloses, polymethacrylates, and starches.

Various useful disintegrants include, but are not limited to, carmellose calcium (Gotoku Yakuhin Co., Ltd.), carboxymethylstarch sodium (Matsutani Kagaku Co., Ltd., Kimura Sangyo Co., Ltd., etc.), croscarmellose sodium (Ac-di-sol™, FMC-Asahi Chemical Industry Co., Ltd.), crospovidones (e.g., Kollidon™ CL manufactured by BASF, Germany), Polyplasdone™ XL, XI-10, and INF-10 (manufactured by ISP Inc., USA), and low-substituted hydroxypropylcelluloses and its different grades such as LH11, LH21, LH31, LH22, LH32, LH20, LH30, LH32 and LH33 (all manufactured by Shin-Etsu Chemical Co., Ltd.), sodium starch glycolate, alginic acid, ammonium calcium alginate, ammonium alginate, calcium alginate, colloidal silicon dioxide, and starches.

Glidants that are useful include colloidal silicon dioxide, talc and combinations thereof.

Useful tablet lubricants include, but are not limited to, magnesium stearate, glyceryl monostearates, palmitic acid, talc, carnauba wax, calcium stearate sodium, sodium or magnesium lauryl sulfate, calcium soaps, zinc stearate, polyoxyethylene monostearates, calcium silicate, silicon dioxide, hydrogenated vegetable oils and fats, stearic acid, and combinations thereof.

Various solvents that can be used in processing include, but are not limited to, water, methanol, ethanol, acidified ethanol, acetone, diacetone, polyols, polyethers, oils, esters, alkyl ketones, methylene chloride, isopropyl alcohol, butyl alcohol, methyl acetate, ethyl acetate, isopropyl acetate, castor oil, ethylene glycol monoethyl ether, diethylene glycol monobutyl ether, diethylene glycol monoethyl ether, dimethylsulphoxide, N,N-dimethylformamide, tetrahydrofuran, and any mixtures thereof.

Some nonlimiting examples of useful antioxidants are butylated hydroxyanisole, butylated hydroxytoluene, ascorbic acid or a salt thereof (e.g., a sodium salt, a calcium salt, a magnesium salt, a potassium salt, a basic amino acid salt, or a meglumine salt), sodium nitrite, sodium hydrogen sulfite, sodium sulfite, a salt of edetic acid (e.g., a sodium salt, a potassium salt, or a calcium salt), erithorbic acid, cysteine hydrochloride, citric acid, cysteine, potassium dichloroisocyanurate, sodium thioglycolate, thioglycerol, sodium formaldehyde sulfoxylate, sodium pyrosulfite, and 1,3-butylene glycol, propyl gallate, and tocopherol or its derivatives.

Other adjuvants may be included, such as coloring agents, sweetening agents, flavoring agents, acidifying agents, basifying agents.

The formulations of the present invention may be coated with a film-forming agent. The coating may be a functional coating or nonfunctional coating.

The term “functional coating” relates to any type of coating that modifies the release of pseudoephedrine or its salts from a dosage form. The rate-controlling polymers as discussed above contribute to the functional coating.

The nonfunctional coating relates to a coating which does not contribute to controlling the release of pseudoephedrine or its salts.

Various useful film-forming agents include, but are not limited to, cellulose or its derivatives including soluble alkyl- or hydroalkyl-cellulose derivatives such as methylcelluloses, hydroxymethyl celluloses, hydroxyethyl celluloses, hydroxypropyl celluloses, hydroxymethyethyl celluloses, hydroxypropyl methylcelluloses, sodium carboxymethyl celluloses, etc., acidic cellulose derivatives such as cellulose acetate phthalates, cellulose acetate trimellitates, and methylhydroxypropylcellulose phthalates, polyvinylacetate phthalates, etc., insoluble cellulose derivatives such as ethylcelluloses and the like, dextrins, starches and starch derivatives, polymers based on carbohydrates and derivatives thereof, natural gums such as gum Arabic, xanthans, alginates, polyacrylic acids, polyvinylalcohols, polyvinylacetates, polyvinylpyrrolidones, polymethacrylates and derivatives thereof (Eudragit™), chitosan and derivatives thereof, shellac and derivatives thereof, and waxes and fat substances.

If desired, the films may contain additional adjuvants for coating processing such as plasticizers, polishing agents, colorants, pigments, antifoam agents, opacifiers, antisticking agents, and the like.

In addition to the above coating ingredients, sometimes pre-formulated coating products will be used, such as those sold as Opadry™, Opadry II Clear 85F19250 (supplied by Colorcon). The products sold in dry form require only dispersing in a liquid before use.

The compositions and/or formulations of the present invention may be prepared using any processing techniques, such as direct compression, wet granulation, dry granulation, fluid bed granulation such as top spray granulation, bottom spray granulation, extrusion and spheronization, drug layering, etc.

An aspect of the present invention further relates to processes for preparing controlled-release pharmaceutical formulations of pseudoephedrine or its salts, wherein embodiments comprise:

1) Sifting drug, and intragranular and extragranular excipients, through a suitable sieve.

2) Dry mixing the sifted drug with intragranular excipients.

3) Dissolving or dispersing a binder in a suitable solvent and using it to granulate the step 2) material.

4) Optionally, extruding the wet mass and spheronizing to form spheroids.

5) Drying the wet granules or spheroids.

6) Sizing the dried granules or spheroids through a sieve.

7) Blending the dried granules or spheroids with sifted extragranular excipients.

8) Blending the step 7 mixture with a lubricant.

9) Compressing the final blend of step 8) into tablets or, alternatively, filling the final blend of step 8) into capsules.

10) Coating the tablets or capsules of step 9) using a coating solution or dispersion, optionally containing drug.

Optionally, steps 3) through 6) can be omitted, and the step 2) materials combined with any desired further components and compressed or filled into capsules. Another alternative to steps 3) through 5) involves roller compacting the step 2) materials and milling the compacted material through a screen to form granules, followed by steps 6) through 10).

Alternatively the drug, together with at least one excipient, may be loaded onto inert cores, different portions of which may be further coated with polymers to form an immediate release fraction and a controlled release fraction.

The formulations are typically packaged in sachets, bottles, unit dose dispensers, containers and closures made of materials such as high-density polyethylene (HDPE), low-density polyethylene (LDPE) and/or polypropylene and/or glass, blisters or strips composed of aluminum or high-density polypropylene, polyvinyl chloride, polyvinylidene dichloride, or aluminum/aluminum foil blisters with a laminated desiccant system. The packages may also comprise various desiccants such as silica gel pouches, molecular sieves, etc.

Equipment suitable for processing the formulations of the present invention include any one or more of rapid mixer granulators, planetary mixers, mass mixers, ribbon mixers, fluid bed processors, extruders, spheronizers, mechanical sifters, blenders, roller compactors, compression machines, rotating bowls or coating pans, tray dryers, fluid bed dryers, rotary cone vacuum dryers, and the like, multimills, fluid energy mills, ball mills, colloid mills, roller mills, hammer mills, and the like.

Pharmaceutical formulations can be subjected to in vitro dissolution evaluation according to Test 711 “Dissolution,” in United States Pharmacopoeia 29, United States Pharmacopeial Convention, Inc, Rockville, Md., 2005 (“USP”), to determine the rate at which the drug is released from the dosage forms, and the content of the drug can be determined in solutions using methods such as high performance liquid chromatography (HPLC).

Certain specific aspects and embodiments of the invention are described in further detail by the examples below, which examples are provided solely for purposes of illustration and should not be construed as limiting the scope of the invention in any manner.

### Example 1

### Pseudoephedrine Hydrochloride 240 mg Extended Release Tablets

| | |
|---|---|
| Ingredient | Quantity |

Core (values are % of core composition)

| | |
|---|---|
| Pseudoepedrine hydrochloride | 36 |
| Hydroxypropyl methylcellulose (K100M CR) | 30 |
| Povidone K-30 | 7.6 |
| Isopropyl alcohol* | q.s. |
| Dicalcium phosphate dihydrate | 20.8 |
| Copovidone (Plasdone ™ S630) | 2 |
| Colloidal silicon dioxide | 1.6 |
| Magnesium stearate | 2 |

Extended Release Coating (values are % of this coating)

| | |
|---|---|
| Ethylcellulose 7 cps | 50 |
| Hydroxypropyl methylcellulose (HPMC 5 cps) | 40 |
| Medium-chain triglycerides (Miglyol ™ 812N) | 10 |
| Isopropyl alcohol* | q.s. |
| Water* | q.s. |

Drug Coating (values are % of this coating)

| | |
|---|---|
| Pseudoephedrine hydrochloride | 70 |
| Hydroxypropyl methylcellulose (HPMC 3 cps) | 30 |
| Water* | q.s. |

*Evaporates during processing.

Manufacturing Process:

Core

1. Pseudoephedrine hydrochloride and hydroxylpropyl methylcellulose are sifted.

2. Granulating fluid is prepared by dissolving povidone in isopropyl alcohol, to form a 10% solution.

3. The mixture of 1 is granulated with the solution of 2.

4. Granules are dried at 50° C. The dried granules are sifted through an ASTM #24 mesh sieve.

5. Extragranular excipients are sifted through an ASTM #40 mesh sieve and mixed with the granules of 4 for about 15 minutes.

6. Magnesium stearate is sifted through an ASTM #40 mesh sieve and blended with the mixture of 5 for about 5 minutes.

7. The lubricated blend of 6 is compressed into tablets having a weight of 500 mg.

Extended Release Coating

8. Ethylcellulose is dissolved in isopropyl alcohol, and HPMC 5 cps is added.

9 Medium-chain triglyceride is dissolved in a small amount of isopropyl alcohol and this solution is added to the solution of 8. Water is added to this to produce a clear solution.

10. Tablets of 7 are coated using the coating solution of 9, to produce a weight gain of 7%, after drying.

Drug Coating

11 HPMC 3 cps is dissolved in water.

12. Pseudoephedrine hydrochloride is added to the solution of 11.

13. The coated tablets of 10 are coated with drug solution of 12.

Tablets prepared as above, and commercial SUDAFED 24 Hour tablets, are subjected to an in vitro dissolution study, with the dissolution conditions: USP Type II apparatus, 50 rpm stirring, 750 mL of 0.1 N HCl for about 1 hour, followed by 250 mL of 0.2 M sodium phosphate tribasic buffer (pH 12). The following table shows the cumulative percentages of drug that are dissolved at various times.

| Hours | SUDAFED | Example 1 |
|---|---|---|
| 1 | 27 | 25 |
| 4 | 35 | 32 |
| 6 | 47 | 38 |
| 8 | 56 | 45 |
| 10 | 66 | 53 |
| 12 | 76 | 61 |
| 16 | 91 | 79 |
| 18 | 96 | 87 |
| 20 | 99 | 94 |
| 24 | 103 | 104 |

### Example 2

### Pseudoephedrine Hydrochloride 240 mg Extended Release Tablets

| | |
|---|---|
| Ingredient | Quantity |

Core (values are % of core composition)

| | |
|---|---|
| Pseudoephedrine hydrochloride | 36 |
| Hydroxypropyl methylcellulose (HPMC K100M CR) | 30 |
| Povidone K30 (PVP K-30) | 7.6 |
| Isopropyl alcohol* | q.s. |
| Mannitol | 13.8 |
| Plasdone S630 | 2 |
| Starch 1500 | 7 |
| Colloidal silicon dioxide | 1.6 |
| Magnesium stearate | 2 |

Extended Release Coating (values are % of this coating)

| | |
|---|---|
| Ethylcellulose 7 cps | 50 |
| Hydroxypropyl methylcellulose (HPMC E5) | 40 |
| Miglyol 812N | 10 |
| Isopropyl alcohol* | q.s. |
| Water* | q.s. |

Drug Coating (values are % of this coating)

| | |
|---|---|
| Pseudoephedrine hydrochloride | 71.4 |
| Povidone K-30 (PVP K-30) | 28.6 |
| Water* | q.s. |

*Evaporates during processing.

Manufacturing process: similar to that of Example 1, except that mannitol and starch 1500 are used instead of dibasic calcium phosphate, in the film coating hydroxypropyl methylcellulose is used instead of povidone, and in the drug coating povidone is used instead of HPMC.

The tablets, and SUDAFED 24 Hour tablets, are subjected to an in vitro dissolution study, using the dissolution conditions of Example 1. The following table shows the cumulative percentages of drug that are dissolved at various times.

| Hours | SUDAFED | Example 2 |
|---|---|---|
| 1 | 26 | 25 |
| 4 | 33 | 28 |
| 6 | 40 | 33 |
| 8 | 48 | 40 |
| 10 | 58 | 51 |
| 12 | 68 | 63 |
| 16 | 82 | 81 |
| 18 | 87 | 88 |
| 20 | 92 | 93 |
| 24 | 97 | 101 |

### Example 3

### Pseudoephedrine Hydrochloride 240 mg Extended Release Tablets

| | |
|---|---|
| Ingredient | Quantity |

Core (values are % of core composition)

| | |
|---|---|
| Pseudoepedrine hydrochloride | 36 |
| Hydroxypropyl methylcellulose (HPMC K100M CR) | 20 |
| Povidone K 30 (PVP K-30) | 7.6 |
| Isopropyl alcohol* | q.s. |
| Dicalcium phosphate dihydrate | 33.2 |
| Plasdone S630 | 2 |
| Colloidal silicon dioxide | 0.25 |
| Magnesium stearate | 1 |

Extended Release Coating (values are % of this coating)

| | |
|---|---|
| Ethylcellulose 7 cps | 50 |
| Hydroxypropyl methylcellulose (HPMC 5 cps) | 40 |
| Miglyol 812N | 10 |
| Isopropyl alcohol* | q.s. |
| Water* | q.s. |

Drug Coating (values are % of this coating)

| | |
|---|---|
| Pseudoephedrine hydrochloride | 66.7 |
| Opadry II Clear 85F19250# | 33.3 |
| Water* | q.s. |

Film Coating (values are % of this coating)

| | |
|---|---|
| Opadry II Clear 85F19250 | 100 |
| Water* | q.s. |

*Evaporates during processing.
#Opadry II Clear 85F19250 contains partially hydrolyzed polyvinyl alcohol, talc, polyethylene glycol (PEG 3350), and polysorbate 80, and is supplied by Colorcon.

Manufacturing Process:

Core

1. Pseudoephedrine hydrochloride and HPMC K100M CR are sifted through a sieve.

2. Granulating fluid is prepared by dissolving PVP K-30 in isopropyl alcohol.

3. The mixture of 1 is granulated with the solution of 2.

4. Granules are dried at 50° C., then are sifted through an ASTM #24 mesh sieve.

5. Extragranular ingredients are sifted through an ASTM #40 mesh sieve and mixed with the granules of 4 for about 15 minutes.

6. Magnesium stearate is passed through an ASTM #60 mesh sieve and blended with the mixture of 5 for about 5 minutes.

7. The lubricated blend of 6 is compressed into tablets having a weight of 500 mg.

Extended Release Coating

8. Ethylcellulose is dissolved in isopropyl alcohol and HPMC 5 cps is dispersed in the solution.

9. Medium-chain triglyceride is dissolved in a small amount of isopropyl alcohol and added to the solution of 8.

10. The tablets of 7 are coated using the solution of 9, to produce a weight gain of 7%, after drying.

Drug Coating

11. Opadry is dispersed in water.

12. Drug is added to 11 and stirred to produce a fine suspension.

13. The tablets of 10 are coated with the suspension of 12.

Film Coating

14. Coating ingredients are added to water and stirred to produce a fine suspension.

15. Tablets of 13 are coated with the suspension of 14, to produce a weight gain of 3%, after drying.

### Example 4

### Pseudoephedrine Hydrochloride 240 mg Extended Release Tablets

| | |
|---|---|
| Ingredient | Quantity |

A. Core (values are % of core composition)
1. Intragranular Component

| | |
|---|---|
| Pseudoepedrine hydrochloride | 36 |
| Hydroxypropyl methylcellulose (HPMC K100M CR) | 20 |

2. Binder

| | |
|---|---|
| Povidone K 30 (PVP K-30) | 7.6 |
| Isopropyl alcohol* | q.s. |

3. Extragranular Component

| | |
|---|---|
| Dicalcium phosphate dihydrate | 33.15 |
| Plasdone S630 | 2 |
| Colloidal silicon dioxide | 0.25 |
| Magnesium stearate | 1 |

B. Extended Release Coating (values are % of this coating)

| | |
|---|---|
| Ethylcellulose 7 cps | 50 |
| Hydroxypropyl methylcellulose (HPMC 5 cps) | 40 |
| Miglyol 812N | 10 |
| Isopropyl alcohol* | q.s. |
| Water* | q.s. |

C. Drug Coating (values are % of this coating)

| | |
|---|---|
| Pseudoephedrine hydrochloride | 66.66 |
| Opadry II Clear 85F19250 | 33.33 |
| Water* | q.s. |

D. Film Coating (values are % of this coating)

| | |
|---|---|
| Opadry II Clear 85F19250 | 100 |
| Water* | q.s. |

*Evaporates during processing.

Manufacturing Process:

Core

1. Pseudoephedrine hydrochloride and HPMC K100M CR are sifted through a sieve.

2. Granulating fluid is prepared by dissolving PVP K-30 in isopropyl alcohol.

3. The mixture of 1 is granulated with the solution of 2.

4. Granules are dried at 50° C., then are sifted through an ASTM #24 mesh sieve.

5. Extragranular ingredients are sifted through an ASTM #40 mesh sieve and mixed with the granules of 4 for about 15 minutes.

6. Magnesium stearate is passed through an ASTM #60 mesh sieve and blended with the mixture of 5 for about 5 minutes.

7. The lubricated blend of 6 is compressed into tablets having a weight of 500 mg.

Extended Release Coating

8. Ethylcellulose is dissolved in isopropyl alcohol, and HPMC 5 cps is dispersed in the solution.

9. Miglyol is dissolved in a small amount of isopropyl alcohol and added to the solution of 8.

10. The tablets of 7 are coated using the solution of 9, to produce a weight gain of 7%, after drying.

Drug Coating

11. Opadry is dispersed in water.

12. Pseudoephedrine hydrochloride is added to 11 and stirred to produce a fine suspension.

13. The tablets of 10 are coated with the suspension of 12.

Film Coating

14. Coating ingredients are added to water and stirred to produce a fine suspension.

15. Tablets of 13 are coated with the suspension of 14, to produce a weight gain of 3%, after drying.

The tablets of Example 4 are packaged in PVC-PVDC blisters or closed HDPE bottles, and are stored for three months at 25° C. and 60% RH, and at 40° C. and 75% RH. Dissolution profiles were obtained using USP Type II apparatus, 50 rpm stirring, 750 mL of 0.1 N HCl for about 1 hour, followed by 250 mL of 0.2 M sodium phosphate tribasic buffer (pH 12). Results for samples of the tablets, before and after storage, and drug assay results (expressed as percentages of the label drug content), are tabulated below.

| | 25° C. and 60% RH | | 40° C. and 75% RH | |
|---|---|---|---|---|
| | HDPE | PVC-PVDC | HDPE | PVC-PVDC |
| Initial | Bottle | Blisters | Bottle | Blisters |

| | |
|---|---|
| Hours | Cumulative % of Drug Dissolved |

Dissolution Profile

| | | | | | |
|---|---|---|---|---|---|
| 1 | 27 | 28 | 29 | 27 | 27 |
| 2 | 29 | 29 | 29 | 29 | 29 |
| 6 | 40 | 43 | 44 | 43 | 44 |
| 12 | 65 | 67 | 68 | 66 | 68 |
| 18 | 87 | 85 | 86 | 84 | 86 |
| 24 | 97 | 94 | 106 | 93 | 105 |

Drug Assay

| | | | | |
|---|---|---|---|---|
| 99.5 | 98.5 | 98.8 | 99.3 | 99.4 |

1. A controlled release formulation comprising pseudoephedrine or a salt thereof, comprising:
I. a core comprising:
a) 10 to 40 percent by weight of pseudoephedrine or a salt thereof;
b) 5 to 30 percent by weight of a water soluble polymer;
c) 1 to 10 percent by weight of a binder;
d) 15 to 40 percent by weight of a water insoluble filler; and
e) optionally, one or more other pharmaceutical excipients; and
II. a coating comprising:
a) 1 to 15 percent by weight of pseudoephedrine or a salt thereof;
b) 1 to 25 percent by weight of a combination of a water soluble polymer and a water insoluble polymer; and
c) 0.1 to 15 percent by weight of a water insoluble plasticizer.

2. The controlled release formulation according to claim 1, wherein pseudoephedrine or a salt thereof is pseudoephedrine hydrochloride.

3. The controlled release formulation according to claim 1, wherein a water soluble polymer comprises a hydroxypropyl methylcellulose, hydroxylpropyl cellulose, or carboxymethyl cellulose.

4. The controlled release formulation according to claim 1, wherein a water soluble polymer comprises a hydroxypropyl methylcellulose.

5. The controlled release formulation according to claim 1, wherein a binder comprises a polyvinylpyrrolidone, acacia, gelatin, guar gum, or a starch.

6. The controlled release formulation according to claim 1, wherein a binder comprises a polyvinylpyrrolidone.

7. The controlled release formulation according to claim 1, wherein a water insoluble filler comprises dibasic calcium phosphate.

8. The controlled release formulation according to claim 1, wherein a water insoluble polymer comprises ethylcellulose, methylcellulose, propylcellulose, ethylmethyl cellulose, or ethylpropyl cellulose.

9. The controller release formulation according to claim 1, wherein a water insoluble plasticizer comprises medium chain triglycerides.

10. The controlled release formulation according to claim 1, which, following immersion into 750 mL of 0.1 N HCl for about 1 hour, then in 250 mL of 0.2 M sodium phosphate tribasic buffer (pH 12), in a USP type II apparatus with 50 rpm stirring, releases contained pseudoephedrine in the cumulative amounts:
i) about 15-30 percent within about 1 hour;
ii) about 20-40 percent within about 4 hours;
iii) about 35-60 percent within about 8 hours;
iv) about 50-70 percent within about 12 hours; and
v) at least about 70 percent within about 20 hours.

11. The controlled release formulation of claim 1, wherein a core comprises granules.

12. The controlled release formulation of claim 11, wherein granules are compressed into a tablet.

13. The controlled release formulation of claim 1, wherein a core is provided with an enteric coating, and a coating comprising pseudoephedrine is applied thereupon.

14. The controlled release formulation of claim 1, wherein a core comprises about 180 mg of pseudoephedrine and a coating comprises about 60 mg of pseudoephedrine.

15. A controlled release formulation comprising pseudoephedrine or a salt thereof, comprising:
I. a core containing about 50-90 percent of a total amount of pseudoephedrine in the formulation, comprising:
f) 10 to 40 percent by weight of pseudoephedrine or a salt thereof;
g) 5 to 30 percent by weight of a water soluble polymer;
h) 1 to 10 percent by weight of a binder;
i) 15 to 40 percent by weight of a water insoluble filler; and
j) optionally, one or more other pharmaceutical excipients; and
II. a coating containing about 10-50 percent of a total amount of pseudoephedrine in the formulation, comprising:
c) 1 to 15 percent by weight of pseudoephedrine or a salt thereof;
d) 1 to 25 percent by weight of a combination of a water soluble polymer and a water insoluble polymer; and
e) 0.1 to 15 percent by weight of a water insoluble plasticizer.

16. The controlled release formulation according to claim 15, wherein pseudoephedrine or a salt thereof is pseudoephedrine hydrochloride.

17. The controlled release formulation of claim 15, wherein a core comprises granules.

18. The controlled release formulation of claim 17, wherein granules are compressed into a tablet.

19. The controlled release formulation of claim 15, wherein a core is provided with an enteric coating, and a coating comprising pseudoephedrine is applied thereupon.

20. The controlled release formulation of claim 15, wherein a core comprises about 180 mg of pseudoephedrine and a coating comprises about 60 mg of pseudoephedrine.
